(19) United States
(12) Patent Application Publication
(10) Pub. No.: US 2005/0110947 A1
(43) Pub. Date: May 26, 2005

(54) Device and method for measuring the diameter of the iridocorneal angle

(76) Inventor: Jean-Paul Chaduc, Clermont-Ferrand (FR)

Correspondence Address: YOUNG & THOMPSON, 745 SOUTH 23RD STREET, 2ND FLOOR, ARLINGTON, VA, 22202, US

(21) Appl. No.: 10/501,728

(22) PCT Filed: Jan. 10, 2003
(86) PCT No.: PCT/FR03/00068

(30) Foreign Application Priority Data
Jan. 16, 2002 (FR) 02/00486

(51) Int. Cl. A61B 3/14; A61B 3/00

(52) U.S. Cl. 351/206

(57) ABSTRACT

A device and method for measuring the iridocorneal angle including: a light source (14) designed to reveal the differences of contrast in an image; image capture unit (10); and image processing system (22) connected to the image capture unit (10). The image processing system (22) receives an image of the eye captured by the image capture unit (10) and supplies in output a value of the iridocorneal angle, the value being obtained based on the analysis of the contrast differences in the image.

The present invention relates to a device for measuring the diameter of the iridocorneal angle and to a corresponding method.

The success of refractive surgery by fitting anterior chamber implants fixed in the iridocorneal angle implies appropriate dimensions of the implant and in particular a good match between the total diameter of the implant (also called the “overall” diameter) and the diameter of the iridocorneal angle formed by the cornea and the periphery of the iris.

Oversizing the implant may lead to deformation thereof, which may be communicated to the iris and to the pupil. This overall deformation of the eye may cause indentation of the haptics of the implant into the root of the iris and be reflected in an inflammatory reaction in the form of goniosynechia and even in encapsulation of the haptics of the implant.

Similarly, undersizing of the implant may allow the displacement of the implant in the eye and consequent loss of endothelial cells if the implant rubs against the endothelium.

Precise determination of the diameter of the iridocorneal angle is therefore important, in particular for choosing the correct implant. However, there is no immediate method of carrying out this determination since the iridocorneal angle is invisible and not directly accessible from outside the eye.

Invasive methods and instruments for measuring the iridocorneal angle are known in the art.

For example, an incision may be made in the eye and a graduated ruler inserted therein until it comes into contact with the iridocorneal angle; the measurement is then read off the ruler.

This type of measurement is generally unreliable and inaccurate. Moreover, an inherent drawback of this invasive type of method and instrument is that the patient has to undergo a surgical operation, which may represent a risk.

Contactless measuring methods are also known in the art.

However, in this case the determination of the angle is generally empirical and a drawback associated with this empirical determination, as with invasive methods and instruments, is the inaccuracy and the associated unreliability of the value obtained.

For example, starting from the corneal diameter, the white-to-white method consists in evaluating the value of the angle by arbitrarily adding from 0.5 mm to 1 mm to the corneal diameter to obtain the overall diameter of the implant to be placed therein.

However, the choice to add a value from 0.5 mm to 1 mm is empirical and is left to the judgement of the practitioner.

Thus the prior art methods and instruments are unreliable and inaccurate, and this may represent a risk for the patient.

The present invention aims to remedy these drawbacks.

To this end, the present invention proposes a device for measuring the iridocorneal angle, characterized in that it comprises:
a light source adapted to produce differences of contrast in an image,
image capture means, and
image processing means connected to said image capture means,
   said image processing means receiving an image of the eye captured by said image capture means and supplying at its output a value of the iridocorneal angle obtained by analyzing contrast differences in said image.

Thus the invention enables the iridocorneal angle of the eye that is to be operated on to be measured accurately during a pre-operative consultation. It therefore reduces the uncertainty and unreliability associated with the choice of the size of the implant. What is more, no surgical operation is necessary, which eliminates all risk to the patient that an operation represents.

In one particular embodiment, the light source emits in the visible spectrum. It is a light-emitting diode, for example.

Thanks to using a light-emitting diode, light is emitted over a wide angle and the emitted light is of high intensity. Moreover, using a component of this kind is relatively simple and the component is one that is routinely used and therefore relatively inexpensive.

In one particular embodiment, the image capture means comprise a CCD video camera.

The images captured by means of a CCD video camera may be processed directly by the image processing means, which saves processing time.

In one particular embodiment, the device further comprises means for triggering image capture that may advantageously take the form of a trigger on the image capture means or a footpedal connected to the image capture means.

The trigger enables the practitioner to be very responsive and to take a snapshot almost instantaneously when he deems that the image, for example displayed on the screen of a personal computer, is correct.

However, operating the trigger may sometimes move the camera and therefore degrade the quality of the image captured. A footpedal avoids this problem, whilst still enabling the practitioner to be highly responsive.

In one particular embodiment, the image processing means comprise a data processing system adapted to execute image analysis software enabling automatic analysis of contrast differences.

In one particular embodiment, the image processing means comprise storage means allowing, for example, archival storage of values of the diameter of the iridocorneal angle and the corresponding snapshots in the form of a database.

In one particular embodiment, the storage means are adapted to store a file containing the captured image.

Accordingly, in the event of a post-operative problem that might linked to the dimensions of the implant, the photograph taken during the pre-operative phase may be consulted to justify the choice that was made as to the overall diameter of the implant.

With the same object as indicated hereinabove, the present invention also proposes a method of operating a device for measuring the iridocorneal angle as succinctly described above, which method is noteworthy in that it comprises the following steps:
the light source is positioned relative to the eye so that the optical axis of the light source forms a predetermined angle of incidence with the main axis of the eye;
an image of the illuminated eye is captured with the image capture means; and
the captured image is processed using contrast differences in the image to determine a value of the iridocorneal angle.

In one particular embodiment, the method further comprises a step of storing the captured image.

In one particular embodiment, during the image processing step, the distance between the geometrical center of the eye and the periphery of the trabecular reflection is measured at a plurality of angular positions.

According to one particular feature, the angle of incidence has a value of 18±2 degrees.

The advantages of the method being similar to those previously indicated of the device for measuring the iridocorneal angle, they are not repeated here.

Other aspects and advantages of the invention will become apparent on reading the following detailed description of one particular embodiment, given by way of non-limiting example.

The description refers to the accompanying drawings, in which:

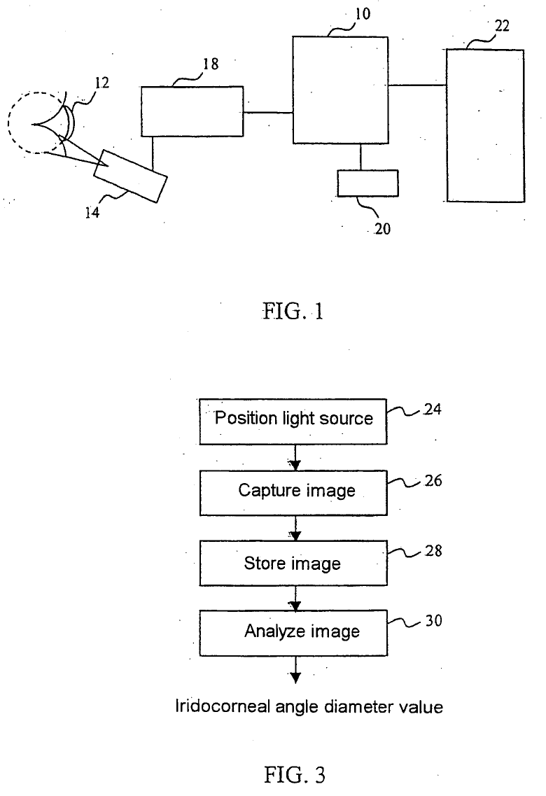

FIG. 1 is a block diagram of one particular embodiment of a device of the invention for measuring the iridocorneal angle;

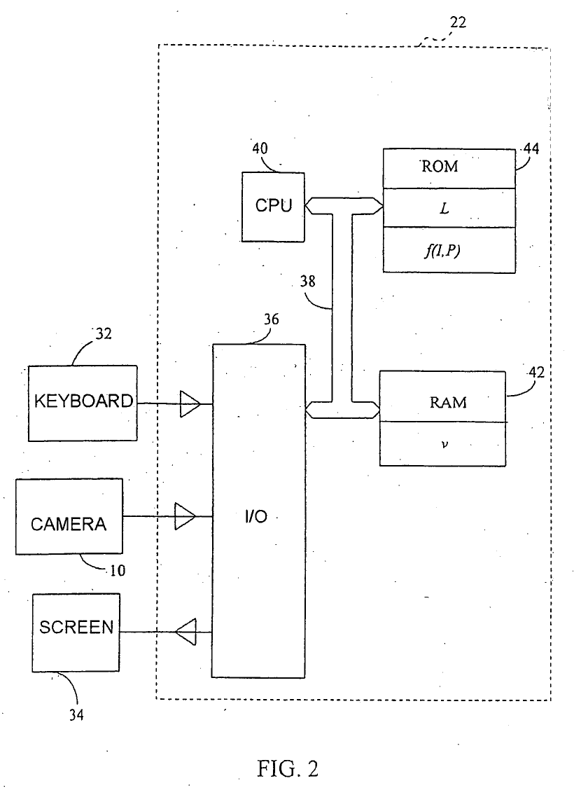

FIG. 2 shows diagrammatically and in more detail the structure of one particular embodiment of a data processing system incorporated into a device of the type shown in FIG. 1; and

FIG. 3 is a flowchart depicting the main steps of one particular embodiment of the method of operating the FIG. 1 device.

Before measuring the iridocorneal angle, the practitioner places the device so that it bears against the orbital floor and seeks the pupillary center using appropriate data processing equipment known in the art, for example by direct display on the screen of a computer. The practitioner also does the focusing.

The diameter of the iridocorneal angle is considered to be symmetrical with respect to the pupillary center determined in this way.

The magnitudes considered are averages given that, firstly, the eye is not perfectly spherical and, secondly, the magnitudes measured are biometric rather than geometric.

For measuring the iridocorneal angle, the present invention is based on analyzing the contrast of the trabecular reflection, the trabeculum being the region situated in the iridocorneal angle.

As shown in FIG. 1, a measuring device according to the invention comprises a camera 10, for example a CCD (charge-coupled device) digital color video camera, facing the eye 12 to be examined. For carrying out the measurements, the eye to be examined may be isolated by placing an eyepiece in front of it.

A light source 14 emitting in the visible spectrum, i.e. in a range of wavelengths from 380 nm to 780 nm, having the property of rendering the sclerotica translucent, provides annular illumination of the iridocorneal angle at grazing incidence relative to the main axis of the eye 12 examined, to illuminate the whole of the cornea and the surrounding region of the sclerotica and the iridocorneal angle. The angle of incidence may be 18°±2°.

This illumination causes a change of contrast in the region of the sclerotica through which the light produced by the light source 14 passes, compared to the portion of the sclerotica through which the light does not pass.

In a preferred embodiment of the invention, the light source 14 is a light-emitting diode (LED), for example a wide-angle high-brightness white LED.

The LED may instead be replaced by a laser diode or by light obtained from optical fibers or a halogen light, this non-exhaustive list being given by way of non-limiting example only.

To facilitate image capture, the gaze of the patient is oriented toward an aiming point provided by a colored light-emitting diode, for example, and mounted at the end of a flexible stalk attached to the video camera, for example.

Images of the illuminated region of the eye are captured by an optical system 18, with no contact with the eye 12. Image capture may be triggered by means of a trigger 20 incorporated in the video camera 10, a footpedal connected to the video camera, or by any other appropriate means.

If the camera 10 is a CCD video camera, the optical system 18 is included in the video camera.

The image capture system may take the form of a pistol integrating the camera 10, the light source 14 fixed at the specific angle of incidence mentioned above, the optical system 18 and the trigger 20, for example.

The camera 10 is connected to a data processing system 22, for example a PC.

As shown in FIG. 2, in one particular embodiment, the data processing system 22 is connected to the camera 10 and to a keyboard 32 and a screen 34 that are together connected to an input/output port 36.

The data processing system 22 comprises, interconnected by an address and data bus 38:
a central processor unit 40;
a random access memory 42;
a read-only memory 44; and
the input/output gate 36.

The components 32, 34, 36, 38, 40, 42 and 44 are well known to the person skilled in the art of microcomputers and therefore are not described here.

The read-only memory 42 is adapted to store in a register “v”, data, variables and intermediate processing results, the term “register” denoting either a memory area of low capacity (able to store a few binary data words) or a memory area of high capacity (able to store an entire program).

The read-only memory 44 is adapted to store the operating program of the central processor unit 40 and, in a register “L”, image analysis software and, in a register “f(I,P)”, one or more files corresponding to the patient(s) examined and containing, for example, the identity and the address of the patient, the captured images, for example in the JPEG (Joint Picture Expert Group) format, and the biometric parameters of the eye or eyes of the patient examined. This information may instead be stored, totally or partly, on a diskette, a CD-ROM or some other storage medium.

The iridocorneal angle is measured by processing the image captured using the image analysis software stored in the register “L”.

The radius or distance between the geometrical center of the eye, as previously determined by the practitioner, and the periphery of the trabecular reflection is measured on the captured image at one or more angular positions, thanks to the contrast observed in this region, which is different from the contrast in the region of the sclerotica through which the light does not pass, as previously indicated.

By way of non-limiting example, measurements at six angular positions equi-angularly distributed over 360 degrees provides for calculating an average radius and thence the diameter of the iridocorneal angle with an uncertainty of only 2%. The accuracy of the resulting measurement is of the order of ±0.1 mm.

FIG. 3 depicts the main steps of the operation of a device of the FIG. 1 type, after the device is switched on.

During a first or positioning step 24 the light source 14 is placed so that its optical axis is at a grazing angle of incidence to the main axis of the eye to be examined. This illuminates an annular region of the eye 12 with the light source 14.

In the next or image capture step 26, after the practitioner has triggered the capture of a picture of the illuminated eye by operating triggering means such as the trigger or the footpedal mentioned above, the camera 10 converts the captured image, which takes the form of a video signal, for example, into a picture file consisting of data bytes. In the conventional way, each picture element (pixel) of the captured image is coded by one or more of these data bytes.

The CCD video camera may be replaced by a conventional video camera and an acquisition card for converting video images supplied by the conventional video camera into digital images, i.e. into the form of data bytes.

The captured image may then be compressed in a manner that is known in the art to store it in the data processing system 22 in the form of a file, for example a bitmap file, in a storage step 28. This step 28 is optional and may be executed after the next or image analysis step 30.

During the image analysis step 30 the red component of the image is extracted first from its red, green and blue components. The red component is particularly advantageous for analyzing contrast variations. Then a minimum contrast variation detection threshold is chosen, as a function of the lighter or darker color of the iris of the eye being examined. This produces a black and white version of the image consisting of the red component previously extracted.

Starting from a previously chosen threshold, a curve illustrating the variation of the luminous contrast is extracted from the black and white image. Peaks on this curve correspond to transition areas between the different regions of the eye. Three central peaks are generally obtained, corresponding to the center and to the contour of the pupil, together with two lateral peaks corresponding to the contours of the trabeculum. The value of the distance between the peak corresponding to the center of the pupil and a peak corresponding to the contour of the trabeculum gives a value of the radius, and therefore of the diameter, of the iridocorneal angle.

The measurement is repeated at different angular positions in order to be able afterwards to calculate an average value of the diameter of the iridocorneal angle.

An average value of the diameter of the iridocorneal angle is obtained in this way. If an anterior chamber implant is to be placed in the eye concerned, the practitioner is easily and reliably able to deduce from this value the appropriate size of the implant to use in the operation.

1. Device for measuring the iridocorneal angle, characterized in that it comprises:
a light source (14) adapted to produce differences of contrast in an image,
image capture means (10), and
image processing means (22) connected to said image capture means (10),
said image processing means (22) receiving an image of the eye captured by said image capture means (10) and supplying at its output a value of the iridocorneal angle obtained by analyzing contrast differences in said image.

2. Device according to claim 1, characterized in that the light source (14) emits in the visible spectrum.

3. Device according to claim 1, characterized in that the light source (14) is a light-emitting diode.

4. Device according to claim 1, characterized in that the image capture means (10) comprise a CCD video camera.

5. Device according to claim 1, characterized in that it further comprises image capture triggering means (20).

6. Device according to claim 5, characterized in that said triggering means (20) comprise a trigger on the image capture means (10).

7. Device according to claim 5, characterized in that said triggering means (20) comprise a footpedal connected to the image capture means (10).

8. Device according to claim 1, characterized in that the image processing means (22) comprise a data processing system adapted to execute image analysis software (L).

9. Device according to claim 8, characterized in that said image processing means (22) comprise storage means (42, 44).

10. Device according to claim 9, characterized in that said storage means are adapted to store a file (f(I,P)) containing said captured image.

11. Method of operating a device for measuring the iridocorneal angle according to any preceding claim 1, characterized in that it comprises the following steps:
the light source (14) is positioned (24) relative to the eye (12) so that the optical axis of the light source (14) forms a predetermined angle of incidence with the main axis of the eye;
an image of the illuminated eye is captured (26) with the image capture means (10); and
the captured image is processed (30) using contrast differences in the image to determine a value of the iridocorneal angle.

12. Method according to claim 11, characterized in that it further comprises a step of storing (28) the captured image.

13. Method according to claim 11, characterized in that, during the image processing step (30), the distance between the geometrical center of the eye and the periphery of the trabecular reflection is measured at a plurality of angular positions.

14. Method according to claim 11, characterized in that the angle of incidence is 18±2 degrees.

15. Device according to claim 2, characterized in that the light source (14) is a light-emitting diode.

16. Device according to claim 2, characterized in that the image capture means (10) comprise a CCD video camera.

17. Device according to claim 3, characterized in that the image capture means (10) comprise a CCD video camera.

18. Method according to claim 12, characterized in that, during the image processing step (30), the distance between the geometrical center of the eye and the periphery of the trabecular reflection is measured at a plurality of angular positions.

19. Method according to claim 12, characterized in that the angle of incidence is 18±2 degrees.

20. Method according to claim 13, characterized in that the angle of incidence is 18±2 degrees.
